(19) United States
(12) Patent Application Publication
(10) Pub. No.: US 2009/0216126 A1
(43) Pub. Date: Aug. 27, 2009

(54) IMPLANTABLE MINIATURIZED ULTRASONIC TRANSDUCERS

(76) Inventors: Lawrence J. Katz, Shaker Heights, OH (US); Dov Hazony, University Heights, OH (US)

Correspondence Address: GREENBERG TRAURIG, LLP, MET LIFE BUILDING, 200 PARK AVENUE, NEW YORK, NY, 10166, US

(21) Appl. No.: 11/913,628

(22) PCT Filed: May 3, 2006
(86) PCT No.: PCT/US06/16894

§ 371 Date: Dec. 8, 2008

Related U.S. Application Data
(60) Provisional application No. 60/677,045, filed on May 3, 2005.

(51) Int. Cl. A61B 8/00

(52) U.S. Cl. 600/449

(57) ABSTRACT

The present invention comprises an ultrasonic measuring device and method for measuring bone resorption and remodeling using Miniaturized Ultrasonic Transducers. An ultrasonic measuring device for insertion into hard tissue comprises an elongate member with an implant end of the elongate member and an acoustic transducer mounted from the interior of the elongate member at the implant end. The implant end of the ultrasonic measuring device is configured such that the acoustic transducer can measure, when inserted into a hard tissue, variations of the hard tissue using ultrasonic wave propagation. The method of taking measurements of bone resorption and remodeling includes taking measurements of a healthy region and remodeling region of a bone using ultrasonic wave propagation; and calculating the variations in the bone's density and its elasticity along the bone's length a) in the healthy region and b) in the remodeling region.

### CROSS REFERENCE TO RELATED APPLICATIONS

This application is a continuation of U.S. Provisional Application No. 60/677,045, filed on May 3, 2005, the entirety of which is incorporated by reference herein.

### BACKGROUND OF THE INVENTION

Every year, in the United States and worldwide, large numbers of surgical procedures are performed in which an instrument, such as a nail, pin, rod, screw, wire, drill bit, or other implant is inserted into a bony tissue mass to stabilize a fracture or defect in such bony tissue mass. The nail or screw strengthens the bone and holds the parts of the bone together. For example, such a technique is used to fix a hip fracture. Hip fracture fixation with either compression hip screws (CHS) or intramedullary interlocking nails is one of the most common orthopedic surgical procedures. The surgeon's goals are accurate reduction and stabilization of the fracture until bony union occurs.

External fixation makes use of such implants during bone remodeling. Remodeling is the cyclical process by which bone maintains a dynamic steady state through sequential resorption and formation of a small amount of bone at the same site; the size and shape of remodeled bone remains unchanged.

### SUMMARY OF THE INVENTION

For purposes of illustration herein, examples regarding the insertion of an implant into the proximal femur of a person will be referred to. This should in no way be interpreted as a limitation on the scope of this invention.

Implantable miniaturized ultrasonic transducers (MUT) have been designed and developed for measurements of bone resorption and remodeling.

In one embodiment is an ultrasonic measuring device comprising an elongate member with an implant end of the elongate member and an acoustic transducer mounted from the interior of the elongate member at the implant end. The device includes an input for electrical power operatively connected to the acoustic transducer. The implant end of the ultrasonic measuring device is configured such that the acoustic transducer can measure, when inserted into a hard tissue, variations of the hard tissue using ultrasonic wave propagation. The acoustic transducer of the ultrasonic measuring device can be mounted from the interior of the elongate member at the implant end through an auxiliary plug. The acoustic transducer can also produce an acoustic beam normal to the elongate member in a controlled direction. Further, the acoustic transducer can be up to about 1 mm in diameter, and can include the capability to operate at a frequency of about 4 MHz.

The elongate member can be configured to operate as an orthopedic implant. Non-limiting examples of implants include a nail, pin, rod, screw, wire, drill bit. The elongate member can also be configured to operate as an external fixator.

In one embodiment, a plurality of external fixators can be configured to be used in combination to take measurements of a healthy region of the hard tissue and a remodeling region of the hard tissue. The configuration of the external fixators can permit self-calibrating measurements of a healthy region of the hard tissue and a remodeling region of the hard tissue.

In another embodiment is an ultrasonic measuring device comprising an elongate member with an implant end of the elongate member, an acoustic transducer mounted from the interior of the elongate member at the implant end, and a cavity on the opposite end of the elongate member from the implant end. The ultrasonic measuring device is configured to allow in vitro measurements of hard tissue formation in the cavity using ultrasonic wave propagation.

In another embodiment is a method of taking measurements of bone resorption and remodeling. The method comprises taking a measurement of a healthy region of a bone using ultrasonic wave propagation and taking a measurement of a remodeling region of a bone using ultrasonic wave propagation. The healthy region measurement and the remodeling region measurement are used to calculate the variations in the bone's density and its elasticity along the bone's length a) in the healthy region and b) in the remodeling region.

### DESCRIPTION OF THE DRAWINGS

FIG. 1 shows MUTs within an insertable screw.

FIG. 2 shows a plurality of external fixators comprising the MUTs as implanted into a fractured femur.

FIGS. 3, 4, and 5 show examples of external fixation of hard tissue.

FIG. 6 shows a graphical representation of results using MUTs for in vitro measurements.

FIG. 7 shows a graphic representation of results using a fast, volumetric method for determining micron and sub-micron particle size in materials as used to scan cast iron.

### DETAILED DESCRIPTION OF THE INVENTION

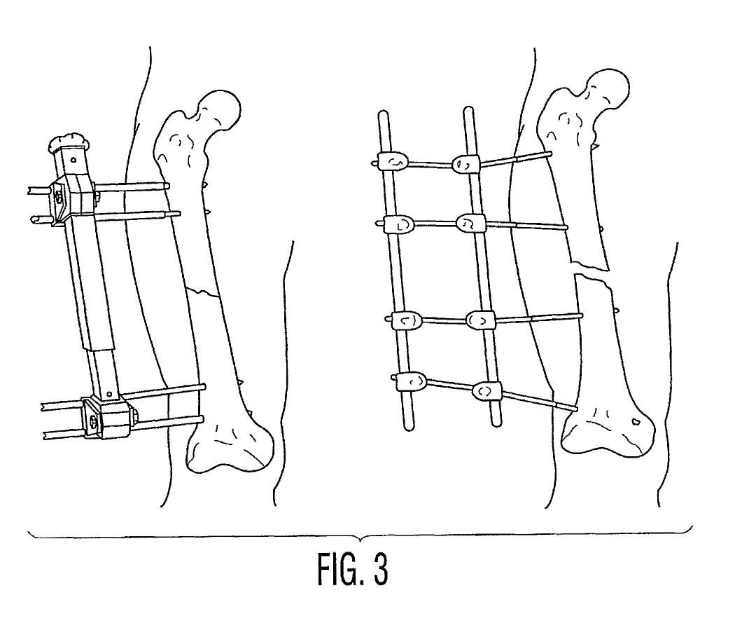

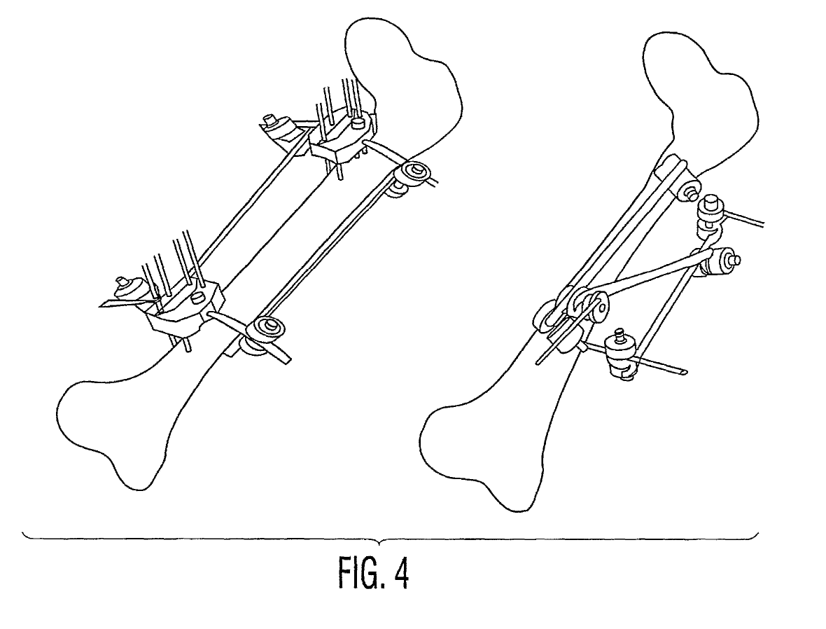

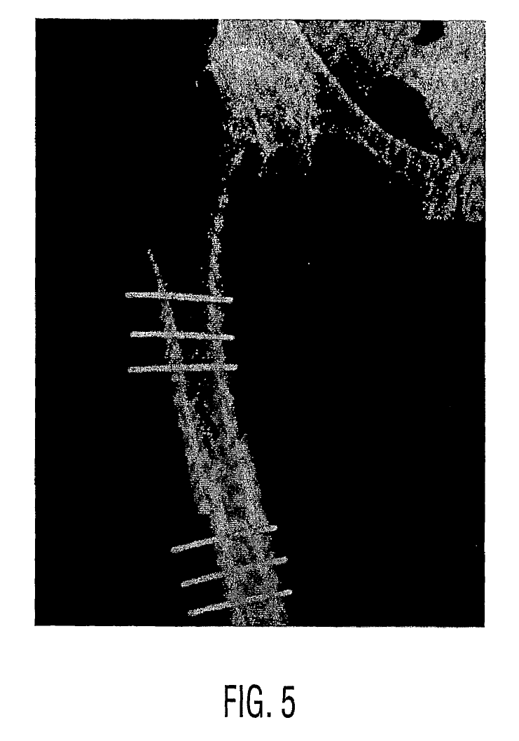

Implantable miniaturized ultrasonic transducers (MUT) have been designed and developed for measurements of bone resorption and remodeling. The measurements can be taken in vivo. The MUT is adapted for inclusion in a fixator, which can be used in external fixation of hard tissue such as bone. External fixation of hard tissue such as bone is used in a number of situations. Temporary external fixation of femoral shaft fractures can be used where there is a patient at the point of death with vascular injuries and severe soft tissue injures. Examples of external fixation of hard tissue (the femur) are shown in FIGS. 3, 4, and 5.

Ultrasonic transducers can be used for ultrasonic wave propagation, which is a non-destructive technique for measuring a material's elastic properties. Signal processing of the acoustic response can be use to obtain measures of other properties such as density and particle size. An example of calculations adapted to measure variations of a bone's density and elastic properties along the length of the bone as well about its circumference can be found in U.S. Pat. No. 5,143,069 entitled “Diagnostic Method of Monitoring Skeletal Defect By In Vivo Acoustic Measurement of Mechanical Strength Using Correlation and Spectral Analysis” to Kwon et al, the entirety of which is incorporated herein. Examples of ultrasonic transducers for use in biomedical and other applications are disclosed in U.S. Pat. No. 5,311,095 entitled “Ultrasonic Transducer Array” to Smith et al., and U.S. Pat. No. 4,907,454 entitled “Ultrasonic Transducer” to Hazony et al., the entirety of each being incorporated herein.

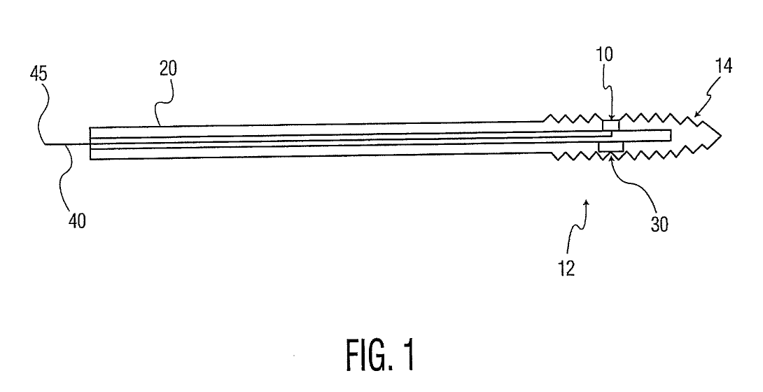

One embodiment of an implantable MUT is shown in FIG. 1. An MUT 10 is adapted for inclusion in an elongate member, the embodiment of the elongate member being an orthopedic screw 12, which can be used as an external fixator. The screw 12 comprises a tube 20. The tube 20 can comprise metal, for example, titanium. The front portion 14 of the screw 12 comprises the MUT 10. The MUT 10 is located so that it can be enclosed within the cortical bone when the screw is implanted.

A transducer 10 up to about 1 mm in diameter is mounted from the inside of a tube 20 through an auxiliary titanium plug 30. An exemplary transducer 10 can be about 1 mm long and at a frequency of about 4 MHz. The transducer 10 is adapted to produce an acoustic beam that is normal to the tubing 20 in a controlled direction. The screw 12 design can be such that that only titanium is exposed to the body. An external electrical drive (not shown) can be provided through the inside of the tubing 20. An input 45 for electrical power can be operatively connected via at least one electrical conductor 40 to the acoustic transducer 10 via the inside of the tube 20. Also, tube 20 may act as an electrical ground.

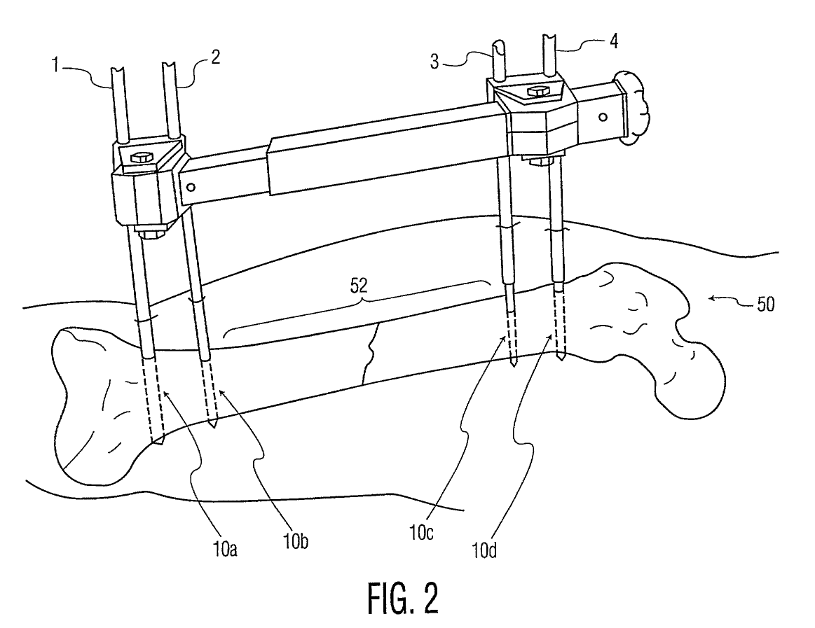

FIG. 2 shows an embodiment of a plurality of elongate members, external fixators 1,2,3,4, comprising the MUTs 10a, 10b, 10c, 10d, which can be implanted into a fractured femur. First and second fixators 1,2 are implanted such that the interval in between the first and second fixators 1,2 span a healthy or normal section of hard tissue 50, shown as femur bone. Similarly, third and fourth fixators 3,4 are implanted such that the interval in between the third and fourth fixators 3,4 span a healthy or normal section of hard tissue 50. The area between the first and third fixators 1,3 spans the remodeling region 52 of the hard tissue 50. The area between the second and fourth fixators 2,4 also spans the remodeling region 52 of the hard tissue. In each fixator 1,2,3,4, is at least one MUT 10a, 10b, 10c, 10d. Using the MUTs 10a, 10b, 10c, 10d, the measurements taken between the first and second fixators 1,2 and the third and fourth fixators 3,4 measure normalized or healthy bone quality. The MUTs 10a, 10b, 10c, 10d measure the remodeling region between the second and fourth fixators 2,4, and the first and third fixators 1,3. This arrangement is self-calibrating as it uses cross-correlation calculations to take into account the variations in a hard tissue's 50 (e.g., the bone's) density and elastic properties along the length of the hard tissue 50.

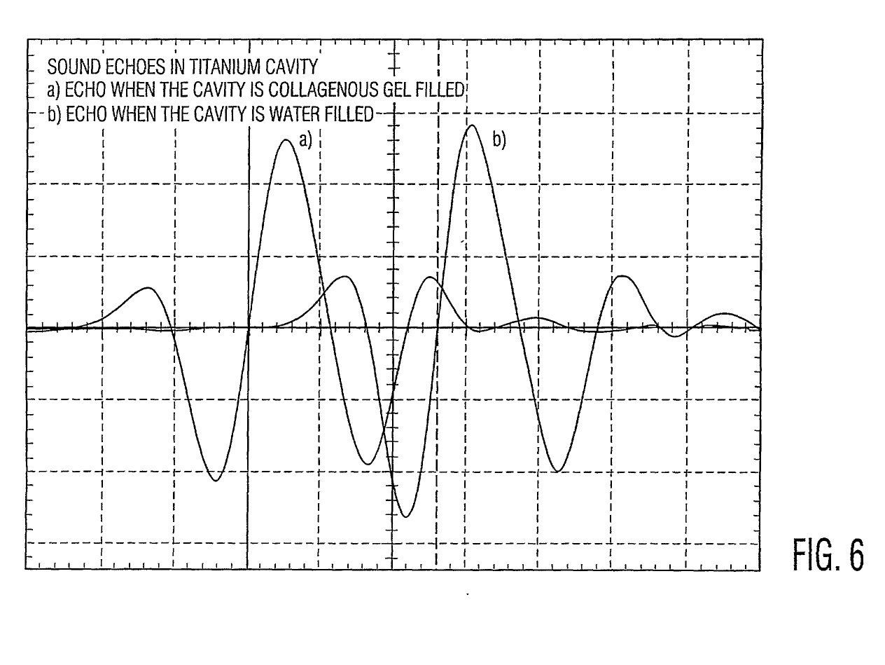

MUTs can be used in animal model studies of bone resorption and remodeling in osteoporosis. Regarding the in vivo use of the implantable screws of present invention using an animal model, external noninvasive measurements of acoustic parameters can be made via a process and device disclosed in the incorporated U.S. Pat. No. 5,143,069. These measurements can be taken simultaneously with the measurements made with the implanted screws. By correlating the two sets of measurements, noninvasive measurements can be used in humans to follow potential changes in bone quality initially measured by the implantable screws. In another embodiment, (not shown) the MUTs may be used for in vitro measurements. In this embodiment, the MUT's have a cavity at one end and the ultrasonic transducer at the other end of a titanium orthopedic screw. Calibration of speed of sound, attenuation, and density in the cavity are done in solutions of graded concentrations of collagen powder in distilled water. In vitro measurements of bone formation in the cavity can then be performed in cell culture experiments. A graphical representation of results using MUTs for in vitro measurements is disclosed in FIG. 6.

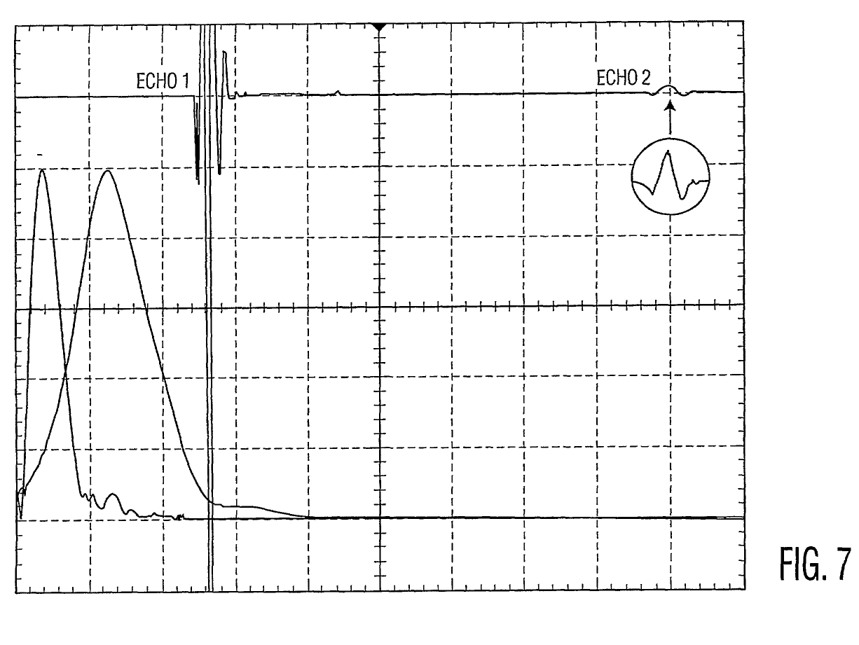

The signal response from in vivo implantable MUTs can be used in conjunction with a method for determining micron and sub-micron particle size limitation in materials without the limitations on resolution proscribed by frequency dependence. A fast, volumetric method for determining micron and sub-micron particle size in materials can be found in “Average Grain-Size Estimation in Polycrystalline Channels” by Hazony, Katz, and Welsch, the entirety of which is incorporated by reference herein, and a graphic representation of results of the method as used to scan cast iron is disclosed in FIG. 7.

It should be understood that the above description is only representative of illustrative embodiments and examples. For the convenience of the reader, the above description has focused on a limited number of representative examples of all possible embodiments, examples that teach the principles of the invention. The description has not attempted to exhaustively enumerate all possible variations or even combinations of those variations described. That alternate embodiments may not have been presented for a specific portion of the invention, or that further undescribed alternate embodiments may be available for a portion, is not to be considered a disclaimer of those alternate embodiments. One of ordinary skill will appreciate that many of those undescribed embodiments, involve differences in technology and materials rather than differences in the application of the principles of the invention. Accordingly, the invention is not intended to be limited to less than the scope set forth in the following claims and equivalents.

1. An ultrasonic measuring device comprising:
an elongate member including and internal and external surface;
an implant end of the elongate member;
an acoustic transducer mounted from the interior of the elongate member at the implant end;
an input for electrical power operatively connected to the acoustic transducer;
whereby the implant end of the ultrasonic measuring device is configured such that the acoustic transducer can measure, when inserted into a hard tissue, variations of the hard tissue using ultrasonic wave propagation.

2. The ultrasonic measuring device of claim 1, wherein the acoustic transducer is mounted from the interior of the elongate member at the implant end through an auxiliary plug.

3. The ultrasonic measuring device of claim 1, wherein the acoustic transducer can produce an acoustic beam normal to the elongate member in a controlled direction.

4. The ultrasonic measuring device of claim 1, wherein the acoustic transducer is up to about 1 mm in diameter.

5. The ultrasonic measuring device of claim 1, wherein the acoustic transducer includes the capability to operate at a frequency of about 4 MHz.

6. The ultrasonic measuring device of claim 1, wherein the elongate member is configured to operate as an orthopedic screw.

7. The ultrasonic measuring device of claim 1, wherein the elongate member is configured to operate as an external fixator.

8. The ultrasonic measuring device of claim 7, further comprising:
a plurality of external fixators;
whereby the plurality of external fixators are configured such that the fixators are capable of taking measurements of a healthy region of the hard tissue and a remodeling region of the hard tissue.

9. The ultrasonic measuring device of claim 8, wherein the configuration of the external fixators permits self-calibrating measurements of a healthy region of the hard tissue and a remodeling region of the hard tissue.

10. An ultrasonic measuring device comprising:
an elongate member;
an implant end of the elongate member;
an acoustic transducer mounted from the interior of the elongate member at the implant end;
a cavity on a non-implant portion of the elongate member;
whereby the device is configured to allow in vitro measurements of hard tissue formation in the cavity using ultrasonic wave propagation.

11. A method of taking measurements of bone resorption and remodeling comprising:
taking a measurement of a healthy region of a bone using ultrasonic wave propagation;
taking a measurement of a remodeling region of a bone ultrasonic wave propagation; and
calculating, using factors comprising the healthy region measurement and the remodeling region measurement, the variations in the bone's density and its elasticity along the bone's length a) in the healthy region and b) in the remodeling region.
